(19) United States
(12) Patent Application Publication
(10) Pub. No.: US 2006/0234887 A1
(43) Pub. Date: Oct. 19, 2006

(54) Soap composition

(75) Inventors: Peter Tollington, Rotterdam (NL); Johannes Helmond, Gouda (NL); J.C.M Helmond-Kuyf, Gouda (NL)

Correspondence Address: MAYER, BROWN, ROWE & MAW LLP, 1909 K STREET, N.W., WASHINGTON, DC, 20006, US

(73) Assignee: Unichema Chemie BV, Gouda (NL)

(21) Appl. No.: 10/531,240

(22) PCT Filed: Oct. 1, 2003
(86) PCT No.: PCT/GB03/04249

§ 371 Date: Mar. 14, 2006

(30) Foreign Application Priority Data
Oct. 14, 2002 (GB) 0223793.1

(51) Int. Cl. A61K 8/02

(52) U.S. Cl. 510/141; 510/153; 510/155

(57) ABSTRACT

A soap composition containing in the range from 60 to 99% by weight of the composition of a mixture of (i) 20 to 80% by weight of alkali metal soap of straight chain C₈-C₂₄ fatty acids, and (ii) 20 to 80% by weight of alkali metal soap of branched C₈-C₂₄ fatty acids, both based on the total weight of alkali metal soaps in the composition. The soap composition exhibits colour and odour stability without the requirement to include any additional stabilizers, preservatives or chelating agents normally present in soap.

### FIELD OF INVENTION

The present invention relates to a soap composition, and in particular to a soap composition comprising soap of branched fatty acids.

### BACKGROUND

A wide range of soap compositions, used to make soap bars or tablets, particularly toilet soap tablets for personal washing, are known in the art. In order for the soap composition to have sufficient stability in storage and in use, it generally contains one or more preservatives, antioxidants or chelating agents. Unfortunately the use of such materials in soaps can lead to certain problems such as sensitization, and contamination of the waste water, particularly due to lack of biodegradability.

A soap composition or soap tablet, is required to have many properties, such as ability to lather, required hardness (suitable for stamping), reduction in mush (softening when left standing in water), pleasant skin feel, and pleasant odour and appearance during the usage life of the soap. It can be difficult to obtain a soap composition having all or most of the aforementioned properties, in particular in a soap composition containing reduced levels (or substantially none at all) of preservatives, antioxidants and/or chelating agents.

### PRIOR ART

U.S. Pat. No. 5,962,382-A is directed to a clear soap bar containing a mixture of sodium and triethanolamine fatty acid soaps, triethanolamine co-solvent, isostearic acid and antioxidant. Isostearic acid is employed to improve the clarity of the soap.

U.S. Pat. No. 4,839,080-A is directed to an antibacterial soap composition comprising iodine.

### SUMMARY OF THE INVENTION

We have now surprisingly discovered a soap composition which overcomes or significantly reduces at least one of the aforementioned problems.

Accordingly, the present invention provides a soap composition comprising in the range from 60 to 99% by weight of the composition of a mixture of (i) 20 to 80% by weight of alkali metal soap of straight chain C₈-C₂₄ fatty acids, and (ii) 20 to 80% by weight of alkali metal soap of branched C₈-C₂₄ fatty acids, both based on the total weight of alkali metal soaps in the composition.

The invention also provides a process of making a soap bar or tablet which comprises extruding a soap composition comprising in the range from 70 to 95% by weight of the composition of a mixture of (i) 20 to 80% by weight of alkali metal soap of straight chain C₈-C₂₄ fatty acids, and (ii) 20 to 80% by weight of alkali metal soap of branched C₈-C₂₄ fatty acids, both based on the total weight of alkali metal soaps in the composition.

The invention further provides a soap tablet or bar comprising in the range from (a) 70 to 95% by weight of a mixture of (i) 20 to 80% by weight of alkali metal soap of straight chain C₈-C₂₄ fatty acids, and (ii) 20 to 80% by weight of alkali metal soap of branched C₈-C₂₄ fatty acids, both based on the total weight of alkali metal soaps in the composition, (b) 0 to 5% by weight of free fatty acids, (c) 0.1 to 1% by weight of salt, (d) 5 to 20% by weight of water, and (e) 0.1 to 2% by weight of polyol.

The invention still further provides the use of alkali metal soap of branched C₈-C₂₄ fatty acids to improve the colour stability and/or odour stability and/or lather volume of a soap composition.

The invention yet further provides the use of alkali metal soap of branched C₈-C₂₄ fatty acids to reduce the amount of stabilizer and/or preservative and/or chelating agent required in a soap composition.

The soap composition according to the present invention may be opaque, translucent or transparent, and is preferably opaque. By “opaque” is meant having the property of preventing the transmission of light so that objects placed behind an opaque soap cannot be seen. By “transparent” is meant having the property of transmitting light without appreciable scattering, so that objects placed behind a transparent soap are entirely visible and can easily be discerned. By “translucent” is meant having the property of allowing light to pass through partially or diffusely so that objects placed behind a translucent soap tablet cannot clearly be distinguished (therefore also called partly transparent or semi-transparent). The amount of light transmitted is, of course, dependent upon the thickness of the soap, and in the present context soap of 20 mm thickness was used as standard.

The soap composition according to the present invention preferably comprises in the range from 65 to 97%, more preferably 70 to 95%, particularly 80 to 92%, and especially 85 to 90% by weight of alkali metal soaps of fatty acids (combination of straight chain and branched fatty acids), based on the total weight of the composition.

The soap composition preferably comprises in the range from 25 to 75%, more preferably 30 to 70%, particularly 35 to 65%, and especially 40 to 60% by weight of alkali metal soap of straight chain or linear C₈-C₂₄, preferably C₁₂-C₂₂ fatty acids; and also preferably comprises in the range from 25 to 75%, more preferably 30 to 70%, particularly 35 to 65%, and especially 40 to 60% by weight of alkali metal soap of branched C₈-C₂₄, preferably C₁₂-C₂₂ fatty acids, both based on the total weight of alkali metal soaps in the composition.

The alkali metal soap straight chain fatty acids preferably comprise greater than 70%, more preferably greater than 80%, particularly greater than 90%, and especially greater than 95%, and up to 100% by weight of C₁₂-C₁₈ fatty acids; and further preferably comprise greater than 50%, more preferably in the range from 55 to 90%, particularly 60 to 80%, and especially 65 to 75% by weight of C₁₆-C₁₈ fatty acids.

The alkali metal soap branched chain fatty acids preferably comprise greater than 70%, more preferably greater than 80%, particularly greater than 90%, and especially greater than 95%, and up to 100% by weight of C₁₆-C₂₂ fatty acids; and further preferably comprise greater than 50%, more preferably in the range from 60 to 90%, particularly 65 to 85%, and especially 70 to 80% by weight of C₁₈ fatty acids.

The alkali metal soap branched chain fatty acids preferably comprise alkyl side branches (attached directly to a carbon atom of the longest linear chain) having on average less than 5, more preferably less than 3, particularly in the range from 1.05 to 2, and especially 1.1 to 1.5 carbon atoms, i.e. the side branches are predominantly methyl groups. In a preferred embodiment of the invention, greater than 50%, more preferably greater than 60%, particularly in the range from 70 to 97%, and especially 80 to 93% by number of the side-branched groups are methyl groups.

In a further preferred embodiment, greater than 30%, more preferably greater than 40%, particularly in the range from 45 to 90%, and especially 50 to 80% by number of alkali metal soap branched chain fatty acids contain single methyl side branches.

Fatty acids, suitable for use herein, can be obtained from natural sources such as, for instance, plant or animal esters (eg palm oil, rape seed oil, palm kernel oil, coconut oil, babassu oil, soybean oil, castor oil, tallow, whale or fish oils, grease, lard, and mixtures thereof). The fatty acids can also be synthetically prepared, for example as described in “Fatty Acids in Industry”, Ed Robert W Johnson, Earl Fritz, Marcel Dekker Inc, 1989 ISBN 0-8247-7672-0. Resin acids, such as those present in tall oil, may be used. Naphthenic acids are also suitable.

At least part of the alkali metal straight chain fatty acid component of a soap composition according to the present invention is preferably derived from palm kernel oil, coconut oil, babassu kernel oil, palm oil, tallow, and/or lard; more preferably from palm kernel oil, and/or coconut oil; and particularly from palm kernel oil. In one preferred embodiment of the invention, the alkali metal straight chain fatty acids are derived from a mixture of palm kernel oil and palm oil.

Suitable branched chain fatty acids for use in the present invention include iso-acids such as isostearic acid, isopalmitic acid, isomyristic acid, isoarachidic acid and isobehenic acid; neo acids such as neodecanoic acid; and other acids such as 2-ethyl hexanoic acid. Isostearic acid is preferred, such as commercially available materials Prisorine 3501, 3502 and 3505 (trade mark, ex Uniqema).

Alkali metal soaps, such as sodium and potassium soaps, can be made by direct saponification of the fats and oils or by the neutralization of the free fatty acids which are prepared in a separate manufacturing process. Particularly preferred in the present invention are the sodium soaps, but small amounts, suitably less than 10%, preferably less than 8%, more preferably less than 5%, and particularly less than 1% by weight of non-sodium soaps, such as potassium soaps, magnesium soaps, ammonium soaps and/or alkanolamine soaps, and especially potassium soaps, may also be present. In a particularly preferred embodiment of the invention, the soap composition comprises substantially no potassium soaps.

In a preferred embodiment of the invention, the alkali metal soap fatty acids suitably comprise less than 10% by weight, preferably less than 5% by weight, more preferably less than 3% by weight, particularly less than 2% by weight, and especially less than 1.5% by weight of unsaturated fatty acids, based on the total weight of alkali metal soaps in the composition. This is suitably achieved by hydrogenating the fatty acids prior to saponification.

The alkali metal soap fatty acids suitably have an iodine value (measured as described herein) of less than 15, preferably less than 10, more preferably in the range from 0.1 to 7, particularly 0.3 to 5, and especially 0.5 to 2 g iodine/100 g.

The alkali metal soap fatty acids suitably have an acid value (measured as described herein) of less than 250, preferably in the range from 180 to 230, more preferably 200 to 225, particularly 210 to 220, and especially 205 to 220 mg KOH/g.

The alkali metal soap fatty acids suitably have a saponification value (measured as described herein) of less than 250, preferably in the range from 180 to 230, more preferably 200 to 225, particularly 210 to 220, and especially 205 to 220 mg KOH/g.

In a particularly preferred embodiment of the present invention, the difference between the acid value and the saponification value of the alkali metal soap fatty acids is preferably less than 10, more preferably less 5, particularly less than 3, and especially less than 2 mg KOH/g.

The soap composition may also comprise a minor amount of one or more synthetic or non-soap detergents, which may be of the anionic, nonionic, amphoteric or cationic type, or mixtures thereof. Preferably less than 25%, more preferably less than 15%, particularly less than 10%, and especially less than 5% by weight, based on the total weight of the composition is non-soap detergent. In a particularly preferred embodiment of the invention, the soap composition comprises substantially no non-soap detergent.

Suitable non-soap detergents include (i) anionic detergents such as the alkyl aryl sulphonates, such as C₁₀-C₂₂ alkyl benzene sulphonates; the olefin sulphonate salts; the C₁₀-C₂₀ paraffin sulphonate salts; the C₈-C₂₂ fatty acyl sarcosinates; the C₈-C₂₂ fatty acyl isethionates and C₈-C₂₂ fatty acyl N-methyl taurides; and C₈-C₂₂ fatty acid alkanol amides; the C₈-C₂₀ alkyl sulphates and the sulphate esters of the reaction product of 1-20 moles of alkylene oxide with 2 to 5 carbon atoms and a saturated straight- or branched-chain aliphatic monohydric C₈-C₂₀ alcohol, such as sodium lauryl ether sulphate, (ii) nonionic detergents such as the reaction products of 1-50 mole of C₂-C₄ alkylene oxide with C₈-C₂₀ primary or secondary alkanols, with dihydric alcohols, and the like, (iii) amphoteric detergents such as the alkyl-β-iminodipropionates, and long-chain imidazole derivatives, such as imidazolinium betaines, and (iv) cationic detergents such as quaternary ammonium compounds, such as stearyl dimethyl benzyl ammonium chloride, and the like.

The concentration of water in the soap composition according to the present invention is preferably in the range from 1 to 25%, more preferably 5 to 20%, particularly 8 to 15%, and especially 10 to 13% by weight based on the total weight of the composition.

The soap composition according to the present invention may also contain free fatty acids, in addition to the neutralized fatty acids of the actual soap component. Preferred free fatty acids are the same types of fatty acids, as defined above, which are used to form the soap component, and therefore generally contain from 8 to 24 carbon atoms. The soap composition suitably comprises in the range from 0 to 10%, preferably 0 to 5%, more preferably 0.5 to 5%, particularly 0.5 to 2%, and especially 0.5 to 1% by weight of free fatty acids, based on the total weight of the composition. The presence of the free fatty acids can improve both the mildness and refatting properties of the soap composition on the skin.

The soap composition suitably comprises less than 2%, preferably less than 1.5%, more preferably in the range from 0.1 to 1%, particularly 0.2 to 0.8%, and especially 0.3 to 0.7% by weight of salt, particularly sodium chloride, based on the total weight of the composition.

The soap composition may also comprise polyols or polyhydric alcohols. Polyols are normally included at a relatively high concentration if a transparent or translucent soap product is required. The concentration of polyol in a transparent or translucent soap composition is preferably in the range from 5 to 20%, more preferably 8 to 18%, particularly 10 to 16%, and especially 12 to 14% by weight based on the total weight of the composition.

The molecular weight of the polyol is preferably less than 300, more preferably in the range from 50 to 270, particularly 80 to 220, and especially 90 to 200.

Suitable polyols include sugar alcohols such as sorbitol, mannitol; (poly)glycols such as (poly)ethylene glycol, (poly)propylene glycol; and other C₃-C₆ polyols containing from 3 to 6 hydroxyl groups such as trimethylolpropane, trimethylolethane, and glycerine. Sugar alcohols, particularly sorbitol, are preferred. Mixtures of any two or more of the aforementioned materials may also be employed, preferably a mixture of comprising a sugar alcohol, particularly sorbitol, and glycerine. The concentration of sugar alcohol is preferably in the range from 1 to 10%, more preferably 3 to 8%, particularly 4 to 7%, and especially 5 to 6% by weight, and the concentration of glycerine is preferably in the range from 1 to 15%, more preferably 3 to 12%, particularly 5 to 10%, and especially 6 to 8% by weight, both based on the total weight of the composition.

The concentration of polyol, preferably glycerine, in an opaque soap composition according to the present invention is preferably less than 5%, more preferably in the range from 0.1 to 2%, particularly 0.5 to 1.5%, and especially 0.8 to 1.2% by weight based on the total weight of the composition.

In order for a soap composition to have the required stability (for example colour stability, odour stability) on storage and/or in use, it is routine practice to incorporate up to 0.5%, normally in the range from 0.05 to 0.1% by weight of one or more additional stabilizers; and/or preservatives, for example antioxidants, such as tocopherols BHA, BHT and the like; and/or chelating agents, such as EDTA (tetra sodium salt of ethylenediaminetetra acetic acid), EHDP (tetra sodium (1-hydroxyethylidene)bisphosphonate) and the like.

A particular surprising feature of the present invention is that the required stability can be achieved without the addition of any of the above mentioned materials. Thus, the soap composition according to the present invention suitably comprises less than 0.5%, preferably less than 0.1%, more preferably less than 0.01%, particularly less than 0.001%, and especially substantially no additional stabilizer and/or preservative and/or chelating agent. The aforementioned ranges are particularly applicable to EDTA and/or EHDP.

The soap composition according to the present invention preferably has a colour stability (or % retained whiteness) (measured as described herein) of greater than 50%, more preferably greater than 60%, particularly in the range from 70 to 95%, and especially 75 to 90%.

The soap composition preferably has an odour stability (measured as described herein) of greater than 50%, more preferably greater than 70%, particularly greater than 80%, and especially in the range from 90 to 100%.

In addition, the soap composition suitably has a total mush value (measured as described herein) of less than 30, preferably in the range from 5 to 25, more preferably 5 to 15, particularly 8 to 12, and especially 6 to 12 g/50 cm².

Another surprising feature of the present invention is that the soap composition exhibits improved lathering, preferably having a lather volume (measured as described herein) of greater than 30, more preferably in the range from 50 to 200, particularly 60 to 150, and especially 80 to 140 ml.

The soap composition may also contain effective amounts of other materials or functional additives. Suitable functional additives include perfumes, emulsifiers such as polyglycerol esters, eg polyglycerol monostearate; colouring agents; deodorants; dyes; emollients and skin conditioners, such as dimerized fatty acids, lanolin, cold cream, mineral oil, sorbitan esters, isopropyl myristate; enzymes; foam stabilizers; lathering agents; moisturizers; optical brighteners; dyes; pearlescers; superfatting agents; UV absorbers; and mixtures of any two or more of these materials. The functional additives may be used in any desired quantity to effect the desired functional characteristics, and usually minor amounts in the range from 0.01 to 5% by weight based on the total weight of the composition are used. For example, if present, (i) emollients and skin conditioning agents generally comprise in the range from 0.5 to about 5% by weight, and (ii) perfumes, dyes and coloring agents comprise in the range from 0.2 to about 5% by weight, all based on the total weight of the composition.

The soap composition according to the present invention may be converted into flakes, noodles, pellets, or any other suitable form or shape by methods known in the art. The converted soap composition, preferably in the form of noodles, can be mixed with other components, such as perfumes, colorants and other functional additives in an amalgamator for at least 5 minutes. The resultant mixture is preferably plodded or extruded, more preferably into an endless bar that, after cutting into billets, can be stamped into a final soap tablet.

The invention is illustrated by the following non-limiting examples.

The following test procedures were employed;

(i) Colour

Colour stability was determined using a compact tristimulus colour analyser (Minolta Chroma meter type CR-300) for measuring reflective colours of surfaces. A pulsed xenon arc lamp in a mixing chamber provided illumination of the sample surface. Six high sensitive silicon photocells, filtered to match the CIE Standard Observer Response, were used by the double beam feed back system of the meter to measure both incident and reflected light. The meter thus detects any slight deviation in the light output by the pulsed xenon arc lamp, and automatically compensates. Absolute measurements were displayed, including Hunter Lab values (L, a, and b). From these values a Whiteness Index was calculated using the formula;
Whiteness Index=(L−3b)

Whiteness Index of the soap tablets was determined both before and after a heat treatment of 16 days in an oven at 50° C., and the difference in whiteness calculated as % of whiteness retained after the heat treatment.

% retained whiteness:

50-70%—moderate
70-80%—good
>80%—excellent
   (ii) Odour

Odour stability was evaluated using a sensory panel of 10 people according to the following method. 125 g of soap composition was placed in a brown coloured 250 ml Scott Duran bottle. The bottle was closed and placed in an oven for 16 days at 50° C. The bottle was then opened and the odour was assessed after cooling of the soap to ambient temperature, using the following scale;
1. Neutral (no rancidity) (100% odour stability (OS))
2. Trace of rancidity (90% OS)
3. Slightly rancid (75% OS)
4. Definitely rancid (medium amount) (50% OS)
5. Strongly rancid (0% OS)

Mean % odour stability values were calculated from the sensory panel assessment of at least 3 bottles.

(iii) Mush

Mush was determined by immersing a well defined portion of a soap tablet (approximate weight=45 g, and approximate surface area=70 cm²) in demineralised water at 20° C. for 2 hours. Before immersion, the weight of the soap block was measured (=W₁). After removal from the water, excess water was allowed to drip from the soap block for 1 minute, and the weight of the soap block was measured again (=W₂). All of the mush was scraped off the soap block with a plastic spatula and the soap block again reweighed (=W₃). The amount of mush is expressed in 3 different parameters, which were calculated according to the following equations (based on an immersed surface area of 50 cm²);
Total mush=(W₂−W₃)×50/immersed area
Water uptake=(W₁−W₂)×50/immersed area
Mushed soap=(W₁−W₃)×50/immersed area

Total mush is a measure of the resistance against slime formation when the soap bar is in contact with water. Water uptake and mushed soap quantity is an indication of measure of the structure of the mush.

Total mush:

<10 g/50 cm²=excellent
11-15 g/50 cm²=good
>15 g/50 cm²=moderate
   (iv) Lather

Lather volume was measured by using a handwash method which closely approximates normal consumer habits. The test was carried out using a pair of surgeon's disposable latex gloves which were rinsed to remove talc. The soap tablets (approximate weight of tablets=85 g, dimensions 8 cm×5.5 cm×2.5 cm (cushion model)) to be tested were washed for 10 minutes before the test by twisting the tablet 20 times through 180° under running water at approximately 14° C. The soap tablet was then immersed in water at a temperature of 20° C., twisted 15 times through 180°, and placed back in the soap dish. Lather was than generated by rubbing the tips of the fingers of one hand against the palm of the other hand 10 times. As much lather as possible was removed from the hands by alternately gripping one hand with the other and forcing lather towards the fingertips. Accumulated lather was dislodged into a 150 ml beaker calibrated at 10 ml intervals. The whole procedure was repeated twice and the total volume of lather recorded as lather volume. Before measuring the lather volume, the lather was gently stirred to release large air pockets. The test was done in triplicate using 3 different soap tablets made from the same composition. Lather volume was calculated as an average value of the three results.

Lather volume:

>100 ml=excellent
50-100 ml=good
<50 ml=moderate
   (v) Rate of Wear

The soap tablets used in the lather volume test were weighed both before and after the test. Weighing after the test was done after the soap had dried at ambient temperature (approximately 23° C.) for at least for 24 hours. The weight difference was recorded as rate of wear and expressed in grams.

Rate of Wear:

<3 g=excellent
3-5 g=good
>5 g=moderate
   (vi) Hardness

A “cheese wire” with an attached weight was cut into the corner of a soap tablet, until an equilibrium position was reached. The area over which the force acts increases as the depth of the cut increases, and therefore the stress being exerted decreases until it is exactly balanced by the resistance of the soap and the wire stops moving. The stress at that point is equal to the yield stress of the soap. The time taken to reach this point was 60 seconds. After this time the weight was removed and the length of the cut measured. The yield stress was calculated from the semi-empirical formula:
Yield stress=⅜(W×98.1)/L×D N/m²
where L and D are the length of the cut and diameter of the wire in cm. W is the weight applied on the wire to obtain the cut and is given in grams.
Hardness:
>7×10⁵ N/m²=hard
>4<7×10⁵ N/m²=moderate
<4×10⁵ N/m²=soft

Hardness is strongly dependent upon temperature and moisture content, and therefore measurements need to be performed under strictly controlled conditions of temperature and moisture (23° C. and 60% relative humidity in this test).

(vii) Iodine Value

The iodine value was determined by the Wijs method (A.O.C.S. Official Method Tg 1-64 (1993)) and expressed as the number of grams of iodine absorbed by 100 grams of sample under the defined test conditions.

(viii) Acid Value

The acid value was measured using the A.O.C.S. Official method Te 1a-64 (Reapproved 1997), and expressed as the number of milligrams of potassium hydroxide required to neutralise the free fatty acids in one gram of sample.

(ix) Saponification Value

The saponification value was determined using the A.O.C.S. Official method Tl 1a-64 (1997) and defined as the number of milligrams of potassium hydroxide which reacts with one gram of sample under the prescribed conditions.

### EXAMPLE 1

A soap composition was prepared containing 17.5% by weight of sodium soap of hydrogenated, topped palm kernel oil fatty acids, 21% by weight of sodium soap of hardened palm oil fatty acids, 49% by weight of sodium soap of isostearic fatty acids (Prisorine 3505 (trade mark, ex Uniqema)), 12% by weight of water, 0.3% by weight of glycerine and 0.4% by weight of sodium chloride. The composition contained 49% by weight of alkali metal soap of straight chain C₁₂-C₂₂ fatty acids; and 50% by weight of alkali metal soap of branched C₁₂-C₂₂ fatty acids, both based on the total weight of alkali metal soaps in the composition. 96% by weight of the alkali metal soap straight chain fatty acids were C₁₂-C₁₈ fatty acids (69% C₁₆-C₁₈); and 99% by weight of the alkali metal soap branched chain fatty acids were C₁₆-C₂₂ fatty acids (76% C₁₈). The soap composition did not contain any additional stabilizers, preservatives or chelating agents normally present in soap.

The soap composition was passed 4 times through a laboratory Mazzoni M-100 duplex refiner/plodder with refining sieves of 1 mm and provided with a rectangular extrusion die of 45 mm×19 mm at the end of the conical outlet. The cylinder temperature was set at 25° C. and the cone temperature was 57° C. The speed of the plodder screw was fixed at 13 rpm. Soap tablets were made from the soap composition produced after 4 passages through the plodder.

### EXAMPLE 2

The procedure of Example 1 was repeated except that the soap composition was prepared containing 15% by weight of sodium soap of hydrogenated, topped palm kernel oil fatty acids, 39% by weight of sodium soap of hardened palm oil fatty acids, 33.4% by weight of sodium soap of isostearic fatty acids, 11% by weight of water, 1% by weight of glycerine and 0.6% by weight of sodium chloride. The composition contained 61.7% by weight of alkali metal soap of straight chain C₁₂-C₂₂ fatty acids; and 38.7% by weight of alkali metal soap of branched C₁₂-C₂₂ fatty acids, both based on the total weight of alkali metal soaps in the composition. The soap composition did not contain any additional stabilizers, preservatives or chelating agents normally present in soap. Soap tablets made from the soap composition were subjected to the test procedures described herein and the results are given in Table 1.

### EXAMPLE 3

The procedure of Example 1 was repeated except that the soap composition was prepared containing 15% by weight of sodium soap of hydrogenated, topped palm kernel oil fatty acids, 44% by weight of sodium soap of hardened palm oil fatty acids, 28.1% by weight of sodium soap of isostearic fatty acids, 11% by weight of water, 1% by weight of glycerine, 0.6% by weight of sodium chloride, and 0.4% by weight of free fatty acids. The composition contained 68% by weight of alkali metal soap of straight chain C₁₂-C₂₂ fatty acids; and 32% by weight of alkali metal soap of branched C₁₂-C₂₂ fatty acids, both based on the total weight of alkali metal soaps in the composition. The soap composition did not contain any additional stabilizers, preservatives or chelating agents normally present in soap. Soap tablets made from the soap composition were subjected to the test procedures described herein and the results are given in Table 1.

### EXAMPLE 4

This is a comparative example not according to the present invention. The procedure of Example 1 was repeated except that the fatty acids were replaced by a standard fatty acid blend commonly used to produce a vegetable soap and containing 17.5% by weight of sodium soap of topped palm kernel oil fatty acids, 70% by weight of sodium soap of palm oil fatty acids, 12% by weight of water, 0.3% by weight of glycerine, and 0.4% by weight of sodium chloride. The soap composition did not contain any additional stabilizers, preservatives or chelating agents normally present in soap. Soap tablets made from the soap composition were subjected to the test procedures described herein and the results are given in Table 1.

TABLE 1

| Properties | Example 2 | Example 3 | Example 4 (Comp) |
|---|---|---|---|
| (i) Colour Stability (%) | 82.0 | 88.4 | 9.5 |
| (ii) Odour Stability (%) | 100 | 100 | 0 |
| (iii) Mush | | | |
| Total mush (g/50 cm²) | 9.9 | 7.2 | 11.6 |
| Water uptake (g/50 cm²) | 5.4 | 3.5 | 8.7 |
| Mushed soap (g/50 cm²) | 4.5 | 3.7 | 2.9 |
| (iv) Lather (ml) | 140 | 140 | 75 |
| (v) Rate of wear (g) | 3.81 | 3.51 | 4.65 |
| (vi) Hardness (×10⁵ N/m²) | 4.32 | 5.19 | 3.62 |

The above examples illustrate the improved properties of a soap composition according to the present invention.

1. A soap composition comprising in the range from 60 to 99% by weight of the composition of a mixture of (i) 20 to 80% by weight of alkali metal soap of straight chain C8-C24 fatty acids, and (ii) 20 to 80% by weight of alkali metal soap of branched CG-C24 fatty acids, both based on the total weight of alkali metal soaps in the composition.

2. A soap composition according to claim 1 comprising 25 to 75% by weight of alkali metal soap of straight chain C8-C24, preferably C12-C22, fatty acids.

3. A soap composition according to claim 1 comprising 25 to 75% by weight of alkali metal soap of branched chain C8-C24, preferably C12-C22, fatty acids.

4. A soap composition according to claim 1 wherein the alkali metal soap straight chain fatty acids comprise greater than 80% by weight of C12-C, 8 fatty acids.

5. A soap composition according to claim 1 wherein the alkali metal soap straight chain fatty acids comprise in the range from 55 to 90% by weight of CIS-CIE fatty acids.

6. A soap composition according to claim 1 wherein the alkali metal soap branched chain fatty acids comprise greater than 70% by weight of C, 6-C22 fatty acids.

7. A soap composition according to claim 1 wherein the branched chain fatty acids comprise isostearic acid.

8. A soap composition according to claim 1 wherein the straight chain fatty acids are derived from palm kernel oil and palm oil.

9. A soap composition according to claim 1 comprising less than 10% by weight of alkali metal soap of unsaturated fatty acids, based on the total weight of alkali metal soaps in the composition.

10. A soap composition according to claim 1 comprising substantially no additional stabilizer and/or preservative and/or cheating agent.

11. A soap composition according to claim 1 having a color stability of greater than 50%.

12. A soap composition according to claim 1 having an odor stability of greater than 50%.

13. A soap composition according to claim 1 in the form of a soap tablet or bar.

14. A process of making a soap bar or tablet which comprises extruding a soap composition comprising in the range from 70 to 95% by weight of the composition of a mixture of (i) 20 to 80% by weight of alkali metal soap of straight chain C8-C24 fatty acids, and (ii) 20 to 80% by weight of alkali metal soap of branched C8-C24 fatty acids, both based on the total weight of alkali metal soaps in the composition.

15. A soap tablet or bar comprising in the range from (a) 70 to 95% by weight of a mixture of (i) 20 to 80% by weight of alkali metal soap of straight chain C8-C24 fatty acids, and (ii) 20 to 80% by weight of alkali metal soap of branched C8-C24 fatty acids, both based on the total weight of alkali metal soaps in the composition, (b) 0 to 5% by weight of free fatty acids, (c) 0.1 to 1% by weight of salt, and (d) 5 to 20% by weight of water, and (e) 0.1 to 2% by weight of polyol.

16. A soap tablet or bar according to claim 15 having at least one or more, and preferably all, of (i) a total mush value in the range from 5 to 25 g/50 cm2, (ii) a lather volume in the range from 60 to 150 ml, (iii) a color stability in the range from 70 to 95%, and (iv) an odor stability of greater than 70%.

17. The use of alkali metal soap of branched C8-C24 fatty acids to improve the color stability and/or odor stability and/or lather volume of a soap composition.

18. The use of alkali metal soap of branched C8-C24 fatty acids to reduce the amount of stabilizer and/or preservative and/or chelating agent required in a soap composition.
